(19) United States
(12) Patent Application Publication
(10) Pub. No.: US 2017/0165175 A1
(43) Pub. Date: Jun. 15, 2017

(54) COSMETIC COMPOSITION FOR TEMPORARY RESHAPING OF KERATINIC FIBERS

(71) Applicant: Henkel AG & Co. KGaA, Duesseldorf (DE)

(72) Inventors: Julia Bibiane Lange, Bad Bramstedt (DE); Bernd Richters, Hamburg (DE); Maria Catalina Bermudez Agudelo, Hamburg (DE); Cyrielle Martinez, Hamburg (DE)

(73) Assignee: Henkel AG & Co. KGaA, Duesseldorf (DE)

(21) Appl. No.: 15/444,117

(22) Filed: Feb. 27, 2017

Related U.S. Application Data
(63) Continuation of application No. PCT/EP2015/064174, filed on Jun. 24, 2015.

(30) Foreign Application Priority Data
Aug. 28, 2014 (DE) 10 2014 217 207.1

(51) Int. Cl. A61K 8/81; A61Q 5/06

(52) U.S. Cl. CPC A61K 8/8152; A61Q 5/06; A61K 2800/48; A61K 2800/594

(57) ABSTRACT

The present invention relates to a cosmetic composition for the temporary shaping of hair, which includes a combination of two specific anionic acrylate resins. The cosmetic composition provides a very good combination of stiffness and long-lasting hold.

### FIELD OF THE INVENTION

The present invention relates to a cosmetic composition for hair setting or for the temporary reshaping of keratinic fibers, in particular human hair, wherein the composition includes a combination of two anionic acrylate resins.

### BACKGROUND OF THE INVENTION

The temporary creation of hairstyles for a longer time period up to a number of days normally requires the use of active setting substances. Hair treatment agents used for a temporary shaping of hair therefore play an important role. Suitable agents for temporary shaping typically include synthetic polymers and/or waxes as an active setting substance. Agents for supporting the temporary reshaping of keratin-containing fibers can be produced, for example, as a hair spray, hair wax, hair gel, or hair foam.

The most important property of an agent for the temporary shaping of hair, also called a styling agent hereinafter, is to give the treated fibers the greatest possible hold in the new modelled shape, i.e., a shape imposed on the hair. This is also referred to as a strong hairstyle hold or a high degree of hold of the styling agent. The hairstyle hold is determined substantially by the type and amount of the employed active setting substances, but the other components of the styling agent may also have an effect.

In addition to a high degree of hold, styling agents can satisfy a wide range of further requirements. These can be divided roughly into properties on the hair, properties of the particular formulation, e.g., properties of the foam, gel, or sprayed aerosol, and properties related to the handling of the styling agent, particular importance being attached to the properties on the hair. Mention can be made in particular of humidity resistance, low tackiness (tack), and a balanced conditioning effect. Furthermore, a styling agent should be universally usable, if possible, for all hair types and be gentle to the hair and skin.

In order to satisfy the different requirements, many synthetic polymers for use in styling agents were already developed as active setting substances. The polymers can be divided into cationic, anionic, nonionic, and amphoteric setting polymers. The polymers when used on hair ideally form a polymer film that, on the one hand, gives the hairstyle a strong hold but, on the other, is sufficiently flexible not to break under stress. If the polymer film is too brittle, so-called film plaques form, i.e., residues that detach as the hair moves and give the impression that the user of the particular styling agent has dandruff. Similar problems arise if waxes are used as the active setting substance in the styling agent. If the styling agent is a gel or a paste, the polymers moreover should have thickening properties.

A known class of anionic polymers, employed in hair setting products, are anionic acrylate resins. EP 1719499 B1, EP 1719500 B 1, and EP 1726331 B1 describe acrylate resins of this type with the INCI name Acrylates/Hydroxy Ester Acrylates Copolymers and their use in styling agents, also in combination with other acrylate copolymers. The anionic acrylate copolymers described in these publications are commercially available, e.g., under the name Acudyne® 180 (The Dow Chemical Company).

WO 2012054278 A2 also mentions Acrylates/Hydroxy Ester Acrylates Copolymers as hair-setting polymers and uses as an example Acudyne® 1000 (The Dow Chemical Company) in hair foams.

Furthermore, hydrophobically modified acrylate copolymers are commercially available (INCI: Acrylates Copolymer (and) Water), which act substantially as thickeners. The data sheet for AquaStyle® SH-100 polymer (Ashland Inc.) describes an acrylate copolymer of this type and the use thereof in combination with carbomers. Suitability for crystal clear hair gels, good initial stiffness, humidity resistance, and a long-lasting effect are described.

### BRIEF SUMMARY OF THE INVENTION

An object of the present invention was to provide other suitable polymer combinations, which are notable for good film-forming and/or setting properties and have a very high degree of hold without having to give up flexibility and good humidity resistance, in particular resistance to sweat and water, and in addition are suitable for producing stable viscous and stable transparent cosmetic compositions. Currently available styling agents in particular can still be improved with respect to the fact that a good combination of stiffness and long-lasting hold is not always sufficiently assured. Therefore, it is an object of the present invention to provide styling agents of this type that, apart from the aforementioned properties, result in particular in both good stiffness and a good long-lasting hold.

This was achieved according to the invention by a combination of two specific anionic acrylate resins.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The cosmetic composition according to one of the preceding points, wherein the composition is present as a hair gel, hair spray, hair foam, or hair wax, in particular as a hair gel.

Use of a cosmetic composition according to one of points 1 to 16 for the temporary reshaping of keratinic fibers.

A method for the temporary shaping of keratinic fibers, in particular human hair, in which the cosmetic composition according to one of points 1 to 16 is applied to keratinic fibers.

In the context of the present invention, it was found surprisingly that an improved combination of a long-lasting effect and stiffness of styling products, in particular of hair gels, can be obtained by the combination of two anionic acrylate copolymers known per se. Such a combination of good stiffness and good long-lasting hold was not expected.

The term ‘keratinic fibers’ according to the invention covers pelts, wool, and feathers but in particular human hair.

The essential components of the cosmetic composition of the invention are the anionic acrylate copolymer (a) and the anionic acrylate copolymer (b).

The anionic acrylate copolymer (a) is made up of at least the following monomer units: at least one (meth)acrylic acid unit (a1), at least one (meth)acrylic acid alkyl ester unit (a2), and at least one (meth)acrylic acid hydroxyalkyl ester unit (a3).

Copolymer (a) according to the invention can be made up of other monomer units. According to embodiments of the invention, copolymer (a), however, is made up only of the units (a1), (a2), and (a3); i.e., it consists of units derived from these monomer units.

The at least one methacrylic acid unit (a1) can be a methacrylic acid or acrylic acid unit.

The alkyl group of the (meth)acrylic acid alkyl ester unit (a2) is preferably a C1-C8 alkyl group, which may be linear or branched. Examples of alkyl groups are methyl, ethyl, propyl, isopropyl, 1 butyl, 2-butyl, isobutyl, tert-butyl, linear or branched pentyl, linear or branched hexyl, linear or branched heptyl, and linear or branched octyl. More preferably, the alkyl group is a C1 to C5 alkyl group. According to an embodiment of the invention, two or more (meth)acrylic acid alkyl ester units (a2) are contained that differ with respect to the carbon number of the alkyl group. For example, a C1-C3 alkyl methacrylate unit and a C2-C5 alkyl acrylate unit are contained.

The hydroxyalkyl group of the (meth)acrylic acid hydroxyalkyl ester unit (a3) can be a hydroxy C1-C10 alkyl group, preferably a hydroxy-C2-C5 alkyl group. In a preferred embodiment, the (meth)acrylic acid hydroxyalkyl ester unit (a3) is a (meth)acrylic acid hydroxyethyl ester.

The proportion of units (a1), (a2), and (a3) in acrylate resin (a) can vary over a broad range. The proportion of unit (a1) in the acrylate copolymer is preferably 2 to 50% by weight, more preferably 5 to 30% by weight. The proportion of unit (a2) in the acrylate copolymer is preferably 5 to 95% by weight, more preferably 45 to 90% by weight. The proportion of unit (a3) in the acrylate copolymer is preferably 2 to 70% by weight, more preferably 5 to 30% by weight.

The weight average of the molecular weight of the anionic acrylate copolymer is preferably 130,000 to 160,000, more preferably 140,000 to 150,000, as determined by gel permeation chromatography (GPC).

The viscosity of the anionic acrylate copolymer (a), used in the cosmetic composition, at a solids content of 44 to 46% by weight and a pH of 3.30 to 4.30 at 25° C. is preferably at most 150 cPS (Brookfield LV, spindle 1, 60 rpm).

Suitable anionic acrylate copolymers (a) are commercially available under the INCI name Acrylates/Hydroxyesters Acrylates Copolymer. The anionic acrylate copolymer (a) Acudyne® 1000 from The Dow Chemical Company is the most preferred. In the commercially available form, it has a solids content of about 44 to 46% by weight and a pH of 3.3-4.3.

Anionic acrylate copolymer (b) is made up of at least the following monomer units: at least one (meth)acrylic acid unit (1)1), at least one (meth)acrylic acid ethyl ester unit (b2), and at least one (meth)acrylic acid ester unit (b3), which is different from the (meth)acrylic acid ethyl ester unit (b2) and has a hydrophobic group as the ester group.

Copolymer (b) according to the invention can be made up of other monomer units. According to embodiments of the invention, copolymer (a), however, is made up only of the units (b1), (b2), and (b3); i.e., it consists of units derived from these monomer units.

The at least one (meth)acrylic acid unit (b 1) can be a methacrylic acid or acrylic acid unit, a methacrylic acid unit being preferred.

The at least one (meth)acrylic acid ethyl ester unit (b2) can be a methacrylic acid ethyl ester unit or an acrylic acid ethyl ester unit, an acrylic acid ethyl ester unit being preferred.

The at least one (meth)acrylic acid ester unit (b3) according to the invention can be a (meth)acrylic acid alkyl ester unit. The alkyl group of the (meth)acrylic acid alkyl ester unit is used to control the hydrophobicity of the copolymer. The alkyl group is preferably a linear or branched alkyl group having 2 to 30 carbon atoms, more preferably 3 to 12 carbon atoms. The hydrophobic group according to the invention can also be a hydrophobic group different from an alkyl group, e.g., an aromatic hydrocarbon ester group. A substituted or unsubstituted phenyl ester group or substituted or unsubstituted alkylene phenyl ester group, e.g., a benzyl ester group, can be cited as an example.

The viscosity of the anionic acrylate copolymer (b), used in the cosmetic composition, at a solids content of 2% by weight and neutralized solution at 25° C. is preferably at most 60,000 to 120,000 cPS.

Suitable anionic acrylate copolymers (a) are commercially available under the INCI name Acrylates Copolymer (and) Water.

Most preferred is the anionic acrylate copolymer (b) AquaStyle® SH-100 polymer from Ashland, Inc. In the commercially available form, it has a solids content of about 28 to 32% by weight and a pH of 2.1-4.0.

The cosmetic composition of the present invention includes acrylate copolymer (a) and acrylate copolymer (b) in amounts which are typical and suitable for styling agents and which can be adapted to the special application and packaging.

The cosmetic composition of the invention includes acrylate copolymer (a), based on the total weight of the cosmetic composition, e.g., in an amount of 0.1 to 5% by weight, preferably 0.1 to 1.5% by weight, more preferably 0.2 to 0.8% by weight, given in each case as the solids content of the active substance in the cosmetic composition.

The cosmetic composition of the invention includes acrylate copolymer (b), based on the total weight of the cosmetic composition, e.g., in an amount of 0.1 to 5% by weight, preferably 0.3 to 2.0% by weight, more preferably 0.8 to 1.4% by weight, given in each case as the solids content of the active substance in the cosmetic composition.

In a particularly preferred embodiment of the present invention, the cosmetic composition includes as anionic acrylate copolymer (a) the copolymer commercially available under the name Acudyne™ 1000 and as anionic acrylate copolymer (b) the copolymer commercially available under the name AquaStyle™ SH-100. Especially good results with respect to a combination of stiffness and long-lasting hold were achieved with this combination. This polymer combination is especially advantageous in styling products in gel form.

Other generally required styling products properties, such as, e.g., humidity resistance and low tackiness, are also achieved particularly with this combination, especially in the case of production as a hair gel.

The cosmetic composition of the present invention preferably includes one or more further component(s), acting as thickeners or gelling agents, which is/are different from acrylate copolymer (a) and (b) and also support film formation. Examples are cationic, anionic, nonionic, or amphoteric polymers. The weight proportion of these further components in terms of the total weight of the cosmetic composition can be relatively low, based on the presence of components (a) and (b), and constitutes, for example, 0.02 to 3% by weight, preferably 0.05 to 1.5% by weight, and even more preferably 0.2 to 0.8% by weight.

Examples are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP Acrylates/C1-18 Alkyl Acrylates/C1-8 Alkyl Acrylamide Copolymer, AMP Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethyl acryl ate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Poly quaternium-10, Polyquaternium-11, Polyquaternium-12, Polyquaternium-13, Po lyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-29, Polyquaternium-30, Po lyquaternium-31, Polyquaternium-32, Polyquaternium-33, Polyquaternium-34, Po lyquaternium-35, Polyquaternium-36, Polyquaternium-37, Polyquaternium-39, Po lyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG 51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate, and Styrene/VP Copolymer.

Examples of nonionic polymers are:

vinylpyrrolidone/vinyl ester copolymers, as they are marketed, for example, under the trademark Luviskol® (BASF). Luviskol VA 64 and Luviskol VA 73, each being vinylpyrrolidone/vinyl acetate copolymers, are preferred nonionic polymers.

cellulose ethers, such as hydroxypropyl cellulose, hydroxyethyl cellulose, and methyl hydroxypropyl cellulose, as they are marketed, for example, under the trademarks Culminal and Benecel (AQUALON).

shellac.

polyvinylpyrrolidones, as they are marketed, for example, under the name Luviskol (BASF).

siloxanes. These siloxanes can be both water-soluble and water-insoluble. Both volatile and nonvolatile siloxanes are suitable, nonvolatile siloxanes being understood to be compounds whose boiling point at normal pressure is above 200° C. Preferred siloxanes are polydialkylsiloxanes such as, for example, polydimethylsiloxane, polyalkylarylsiloxanes such as, for example, polyphenylmethylsiloxane, ethoxylated polydialkylsiloxanes, and polydialkylsiloxanes that include amine and/or hydroxy groups.

glycosidically substituted silicones.

The further component acting as a gelling agent is preferably a homopolyacrylic acid (INCI: Carbomer), which is commercially available under the name Carbopol® in various versions. The carbomer is preferably contained in a proportion of 0.02 to 3% by weight, preferably 0.05 to 1.5% by weight, and even more preferably 0.2 to 0.8% by weight, in regard to the total weight of the cosmetic composition.

The cosmetic composition of the invention can include other conventional styling product substances. Additional care substances can be named in particular as other suitable auxiliary substances and additives.

The agent can include as a care substance, for example, at least one protein hydrolysate and/or a derivative thereof. Protein hydrolysates are product mixtures obtained by acid-, base-, or enzyme-catalyzed degradation of proteins. The term ‘protein hydrolysates’ according to the invention is also understood to mean total hydrolysates, as well as individual amino acids and derivatives thereof, and mixtures of different amino acids. The molar weight of protein hydrolysates usable according to the invention is between 75 (the molar weight of glycine) and 200,000; the molar weight is preferably 75 to 50,000 daltons and very particularly preferably 75 to 20,000 daltons.

The agent of the invention can include further at least one vitamin, provitamin, vitamin precursor, and/or a derivative thereof as a care substance. In this case, the vitamins, provitamins, and vitamin precursors that are usually assigned to the groups A, B, C, E, F, and H are preferred according to the invention.

Like the addition of glycerol and/or propylene glycol, the addition of panthenol increases the flexibility of the polymer film formed during use of the agent of the invention.

The agents of the invention, furthermore, can include at least one plant extract but also mono- or oligosaccharides and/or lipids as a care substance.

Furthermore, oil components are suitable as a care substance. Natural and synthetic cosmetic oil components include, for example, plant oils, liquid paraffin oils, isoparaffin oils, and synthetic hydrocarbons, as well as di-n-alkyl ethers with a total of 12 to 36 C atoms, especially 12 to 24 C atoms. Preferred cosmetic agents of the invention include at least one oil component, preferably at least one oil component from the group of silicone oils. The group of silicone oils comprises in particular dimethicones, which also include cyclomethicones, amino-functional silicones, as well as dimethiconols. Dimethicones can be both linear and branched, as well as cyclic or cyclic and branched. Suitable silicone oils or silicone gums are, in particular, dialkyl- and alkylarylsiloxanes, such as, for example, dimethylpolysiloxane and methylphenylsiloxane, as well as alkoxylated, quaternized, or also anionic derivatives thereof. Cyclic and linear polydialkylsiloxanes, alkoxylated and/or aminated derivatives thereof, dihydroxypolydimethyl siloxanes, and polyphenylalkylsiloxanes are preferred.

Further preferred nourishing oil components are ester oils, i.e., esters of C6-C30 fatty acids with C2-C30 fatty alcohols, preferably monoesters of fatty acids with alcohols having 2 to 24 C atoms, such as, for example, isopropyl myristate (Rilanit® IPM), isononanoic acid C16-18 alkyl ester (Cetiol® SN), 2-ethylhexyl palmitate (Cegesoft® 24), stearic acid-2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V).

Suitable as care substances, furthermore, are dicarboxylic acid esters, symmetric, asymmetric, or cyclic esters of carbonic acid with fatty alcohols, tri-fatty acid esters of saturated and/or unsaturated, linear and/or branched fatty acids with glycerol, or fatty acid partial glycerides, which are understood to be monoglycerides, diglycerides, and technical mixtures thereof.

Furthermore, the composition of the invention preferably includes emulsifiers or surface-active agents. Preferred are PEG derivatives of hydrogenated castor oil, which can be obtained, e.g., under the name PEG Hydrogenated Castor Oil, e.g., PEG-30 Hydrogenated Castor Oil, PEG-33 Hydrogenated Castor Oil, PEG-35 Hydrogenated Castor Oil, PEG-36 Hydrogenated Castor Oil, or PEG-40 Hydrogenated Castor Oil. The use of PEG-40 Hydrogenated Castor Oil is preferred according to the invention. These are contained preferably in an amount of 0.05 to 1.5% by weight, more preferably 0.1 to 1.0% by weight, likewise preferably 0.2 to 0.8% by weight, or 0.3 to 0.6% by weight.

The cosmetic agents of the invention include the ingredients or active substances in a cosmetically acceptable carrier.

Preferred cosmetically acceptable carriers are aqueous, alcoholic, or aqueous-alcoholic media with preferably at least 10% by weight of water, based on the total weight of the agent.

Particularly preferably, the cosmetic carrier of the invention includes water, particularly in the amount that the cosmetic agent includes, based on the total weight of the agent, at least 10% by weight, in particular at least 20.0% by weight, most preferably at least 40% by weight of water.

In particular, lower alcohols, usually used for cosmetic purposes and having 1 to 4 carbon atoms, such as, for example, ethanol and isopropanol, can be contained as alcohols.

Examples of water-soluble solvents as cosolvent are glycerol and/or ethylene glycol and/or 1,2-propylene glycol in an amount of 0 to 30% by weight, based on the total agent.

The cosmetic composition of the present invention can be produced in forms typical for the temporary reshaping of hair, e.g., as a hair gel, hair spray, hair foam, or hair wax. Production as a hair gel is preferred.

Both hair foams and hair sprays require the presence of propellants. According to the invention, however, no or only small amounts of hydrocarbons should be used for this. Dimethyl ether is a suitable propellant according to the invention.

The present invention also relates to the use of cosmetic compositions of the invention for the temporary reshaping of keratinic fibers, in particular of human hair, and to a method for the temporary shaping of keratinic fibers, in particular human hair, in which the cosmetic composition of the invention is applied to keratinic fibers.

Overview table: The composition of some preferred cosmetic agents can be obtained from the following tables (data are given in percentages by weight, based on the total weight of the cosmetic agent, unless otherwise stated).

| | Formula 1 | Formula 2 | Formula 3 | Formula 4 | Formula 5 |
|---|---|---|---|---|---|
| Copolymer a) | 0.1 to 5.0 | 0.1 to 3.5 | 0.2 to 1.5 | 0.3 to 0.8 | 0.4 to 0.6 |
| Copolymer b) | 0.1 to 5.0 | 0.2 to 4.0 | 0.3 to 2.0 | 0.6 to 1.4 | 0.8 to 1.2 |
| Misc. | To 100 | To 100 | To 100 | To 100 | To 100 |

| | Formula | Formula | Formula | Formula | Formula |
|---|---|---|---|---|---|
| | 1a | 2a | 3a | 4a | 5a |
| Copolymer a): | 0.1 to 5.0 | 0.1 to 3.5 | 0.2 to 1.5 | 0.3 to 0.8 | 0.4 to 0.6 |
| Acrylates/ | | | | | |
| Hydroxyesters | | | | | |
| Acrylates | | | | | |
| Copolymer | | | | | |
| Copolymer b): | 0.1 to 5.0 | 0.2 to 4.0 | 0.3 to 2.0 | 0.6 to 1.4 | 0.8 to 1.2 |
| Acrylates | | | | | |
| Copolymer | | | | | |
| (and) Water | | | | | |
| Misc. | To 100 | To 100 | To 100 | To 100 | To 100 |

| | Formula | Formula | Formula | Formula | Formula |
|---|---|---|---|---|---|
| | 1b | 2b | 3b | 4b | 5b |
| Copolymer a): | 0.1 to 5.0 | 0.1 to 3.5 | 0.2 to 1.5 | 0.3 to 0.8 | 0.4 to 0.6 |
| Acudyne ® | | | | | |
| 1000 | | | | | |
| Copolymer b): | 0.1 to 5.0 | 0.2 to 4.0 | 0.3 to 2.0 | 0.6 to 1.4 | 0.8 to 1.2 |
| AquaStyle ® | | | | | |
| SH-100 | | | | | |
| Misc. | To 100 | To 100 | To 100 | To 100 | To 100 |

| | Formula | Formula | Formula | Formula | Formula |
|---|---|---|---|---|---|
| | 11 | 12 | 13 | 14 | 15 |
| Copolymer | 0.1 to 5.0 | 0.1 to 3.5 | 0.2 to 1.5 | 0.3 to 0.8 | 0.4 to 0.6 |
| a) | | | | | |
| Copolymer | 0.1 to 5.0 | 0.2 to 4.0 | 0.3 to 2.0 | 0.6 to 1.4 | 0.8 to 1.2 |
| b) | | | | | |
| Carbomer | 0.02 to 3.0 | 0.05 to 2.0 | 0.05 to 1.5 | 0.2 to 1.0 | 0.4 to 0.8 |
| Misc. | To 100 | To 100 | To 100 | To 100 | To 100 |

| | Formula | Formula | Formula | Formula | Formula |
|---|---|---|---|---|---|
| | 11a | 12a | 13a | 14a | 15a |
| Copolymer a): | 0.1 to 5.0 | 0.1 to 3.5 | 0.2 to 1.5 | 0.3 to 0.8 | 0.4 to 0.6 |
| Acrylates/ | | | | | |
| Hydroxyesters | | | | | |
| Acrylates | | | | | |
| Copolymer | | | | | |
| Copolymer b): | 0.1 to 5.0 | 0.2 to 4.0 | 0.3 to 2.0 | 0.6 to 1.4 | 0.8 to 1.2 |
| Acrylates | | | | | |
| Copolymer | | | | | |
| (and) Water | | | | | |
| Carbomer | 0.02 to 3.0 | 0.05 to 2.0 | 0.05 to 1.5 | 0.2 to 1.0 | 0.4 to 0.8 |
| Misc. | To 100 | To 100 | To 100 | To 100 | To 100 |

| | Formula | Formula | Formula | Formula | Formula |
|---|---|---|---|---|---|
| | 11b | 12b | 13b | 14b | 15b |
| Copolymer a): | 0.1 to 5.0 | 0.1 to 3.5 | 0.2 to 1.5 | 0.3 to 0.8 | 0.4 to 0.6 |
| Acudyne ® | | | | | |
| 1000 (data | | | | | |
| given as the | | | | | |
| solids content) | | | | | |
| Copolymer b): | 0.1 to 5.0 | 0.2 to 4.0 | 0.3 to 2.0 | 0.6 to 1.4 | 0.8 to 1.2 |
| AquaStyle ® | | | | | |
| SH-100 (data | | | | | |
| given as the | | | | | |
| solids content) | | | | | |
| Carbomer | 0.02 to 3.0 | 0.05 to 2.0 | 0.05 to 1.5 | 0.2 to 1.0 | 0.4 to 0.8 |
| Misc. | To 100 | To 100 | To 100 | To 100 | To 100 |

| | Formula | Formula | Formula | Formula | Formula |
|---|---|---|---|---|---|
| | 21 | 22 | 23 | 24 | 25 |
| Copolymer a) | 0.1 to 5.0 | 0.1 to 3.5 | 0.2 to 1.5 | 0.3 to 0.8 | 0.4 to 0.6 |
| Copolymer b) | 0.1 to 5.0 | 0.2 to 4.0 | 0.3 to 2.0 | 0.6 to 1.4 | 0.8 to 1.2 |
| Carbomer | 0.02 to 3.0 | 0.05 to 2.0 | 0.05 to 1.5 | 0.2 to 1.0 | 0.4 to 0.8 |
| PEG-40 | 0.05 to 1.0 | 0.1 to 1.0 | 0.2 to 0.9 | 0.3 to 0.8 | 0.4 to 0.6 |
| Hydrogenated | | | | | |
| Castor Oil | | | | | |
| Misc. | To 100 | To 100 | To 100 | To 100 | To 100 |

| | Formula | Formula | Formula | Formula | Formula |
|---|---|---|---|---|---|
| | 21a | 22a | 23a | 24a | 25a |
| Copolymer a): | 0.1 to 5.0 | 0.1 to 3.5 | 0.2 to 1.5 | 0.3 to 0.8 | 0.4 to 0.6 |
| Acrylates/ | | | | | |
| Hydroxyesters | | | | | |
| Acrylates | | | | | |
| Copolymer | | | | | |
| Copolymer b): | 0.1 to 5.0 | 0.2 to 4.0 | 0.3 to 2.0 | 0.6 to 1.4 | 0.8 to 1.2 |
| Acrylates | | | | | |
| Copolymer | | | | | |
| (and) Water | | | | | |
| Carbomer | 0.02 to 3.0 | 0.05 to 2.0 | 0.05 to 1.5 | 0.2 to 1.0 | 0.4 to 0.8 |
| PEG-40 | 0.05 to 1.5 | 0.1 to 1.0 | 0.2 to 0.9 | 0.3 to 0.8 | 0.4 to 0.6 |
| Hydrogenated | | | | | |
| Castor Oil | | | | | |
| Misc. | To 100 | To 100 | To 100 | To 100 | To 100 |

| | Formula | Formula | Formula | Formula | Formula |
|---|---|---|---|---|---|
| | 21b | 22b | 23b | 24b | 25b |
| Copolymer a): | 0.1 to 5.0 | 0.1 to 3.5 | 0.2 to 1.5 | 0.3 to 0.8 | 0.4 to 0.6 |
| Acudyne ® | | | | | |
| 1000 (data | | | | | |
| given as the | | | | | |
| solids content) | | | | | |
| Copolymer b): | 0.1 to 5.0 | 0.2 to 4.0 | 0.3 to 2.0 | 0.6 to 1.4 | 0.8 to 1.2 |
| AquaStyle ® | | | | | |
| SH-100 (data | | | | | |
| given as the | | | | | |
| solids content) | | | | | |
| Carbomer | 0.02 to 3.0 | 0.05 to 2.0 | 0.05 to 1.5 | 0.2 to 1.0 | 0.4 to 0.8 |
| PEG-40 | 0.05 to 1.5 | 0.1 to 1.0 | 0.2 to 0.9 | 0.3 to 0.8 | 0.4 to 0.6 |
| Hydrogenated | | | | | |
| Castor Oil | | | | | |
| Misc. | To 100 | To 100 | To 100 | To 100 | To 100 |

According to the invention, “misc.” is understood to be a cosmetic carrier, in particular water and optionally other conventional styling product components.

### EXAMPLES

1. The following styling gel was prepared:

| Component/raw | % by | | |
|---|---|---|---|
| material | weight | INCI name | Manufacturer |
| Water, demineralized | 61.476 | | |
| Polygel HP/Synthalen HP | 0.6 | Carbomer | 3V |
| DMDM Hydantoin 55% | 0.1 | DMDM Hydantoin | |
| Water, demineralized | 2.754 | | |
| Sodium hydroxide pearls | 0.49 | | |
| Water, demineralized, | 29.7 | | |
| without H₂O₂ | | | |
| Acudyne ® 1000 | 1 | Acrylates/ | Dow |
| Polymer | | Hydroxyesters | |
| | | Acrylates Copolymer | |
| AquaStyle SH-100 | 3.3 | Acrylates Copolymer | Ashland |
| | | (and) Water | Inc. |
| Castor oil hydrog., 40 EO | 0.4 | PEG-40 | |
| | | Hydrogenated | |
| | | Castor Oil | |
| Water, demineralized | 0.1 | | |
| Perfume TEU-D-4106/6 | 0.08 | | |
| Nutri Acai | | | |
| Total | 100 | | |

The quantitative data in the table are given in percentages by weight of the particular raw material, based on the total composition.

Upon application to human hair, the hair gel forms a transparent film with a very low tackiness.

Stiffness:

850 mg of a gel-like test composition was massaged with the fingers into dry hair strands (Euro-Natural Hair from the company Kerling, 826500 glued braid dense, glued on one side, total length 150 mm, free length 130 mm, width 20 mm, weight 1.8±0.2 g). The hair strand treated with the test composition to be tested was straightened in a Teflon-coated bar with a diameter of 20 mm. The prepared strands were then dried overnight at 21° C. and 50% relative humidity in the environmental chamber and conditioned.

The conditioned strand was carefully removed from the Teflon-coated bar. The resulting flat strand was placed on the measuring blocks removed by 40 mm from one another. The 3PB adapter of a universal testing instrument AMETEK LF Plus from the company AMETEK Precision Instruments Europe GmbH, Product Group Lloyd, was mounted in the middle above this. The entire measurement took place in the environmental chamber under constant climatic conditions at 21° C. and 50% relative humidity.

So as to create standardized initial conditions, the measurement began with the application of a preload of 0.05 N. Next, the strand was compressed 15 mm at a rate of 500 mm min-1, the force necessary for this being measured. Then, the characteristic force K at the maximum deformation of 15 mm was recorded.

Based on the force Fmax as a parameter, the stiffness of the polymer film and the degree of hold of the styling hold generated thereby can be measured using this measuring method.

Ten strands were prepared and measured per test compositions. Stiffness values in the range of 3.0 to 3.5 N were obtained, wherein this refers to arithmetic averages.

Long-lasting hold:

The composition was tested by a long-lasting hold measurement with respect to their shaping properties. For this purpose, standardized hair strands from the company

Kerling (Item No. 826500) of the hair type “European Natural,” color 6/0, with a length (Lmax) of 220 mm and a weight of 3.0 g were used. For preparation, the strands were washed with a 12.5% by weight sodium laureth sulfate solution. The hair strands were dried overnight in a drying oven at 318 K.

The hair was soaked for 20 minutes in lukewarm water and then blotted to about 50% residual moisture in the hair.

750 mg of the composition was applied to each hair strand and massaged in. The hair strands were placed in a Teflon-coated bar, smoothed with a steel roll, and dried overnight at 21° C. and 50%.

The hair strands were then fixed at one of their ends in a holding device and stored for a period of 6 hours at 21° C. and 85% relative humidity. To calculate the long-lasting hold (LLH), the strand length protruding from the holding device was measured before (LO) and after (Lt) the storage.

The long-lasting hold is a measure for the change in length over time of a hair strand fixed by a hair shaping agent. The higher the LLH value, the smaller the change in length of the hair strands under the effect of humidity within a specific time period and the better the degree of hold of the hair shaping agent.

The long-lasting hold was calculated using the following formula.

LLH =1−(Lt−L0/Lmax)

An LLH value of 59 to 61% was determined (arithmetic average of the LLH values of 10 test strands).

The gel of the invention therefore revealed an exceptionally good combination of long-lasting hold and stiffness.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1) A cosmetic composition for the temporary shaping of keratinic fibers, comprising:
a. at least one anionic acrylate copolymer (a), which includes at least the following monomer units: at least one (meth)acrylic acid unit (a1), at least one (meth)acrylic acid alkyl ester unit (a2), and at least one (meth)acrylic acid hydroxyalkyl ester unit (a3), and
b. at least one anionic acrylate copolymer (b), which includes at least the following monomer units: at least one (meth)acrylic acid unit (b1), at least one (meth)acrylic acid ethyl ester unit (b2), and at least one (meth)acrylic acid ester unit (b3), which is different from the (meth)acrylic acid ethyl ester unit (b2) and has a hydrophobic group as the ester group.

2) The cosmetic composition according to claim 1, wherein the anionic acrylate copolymer (a) has a molecular weight of 130,000 to 160,000.

3) The cosmetic composition according to claim 1, wherein the anionic acrylate copolymer (a) has hydroxyethyl (meth)acrylate as monomer unit (a3).

4) The cosmetic composition according to claim 1, wherein the anionic acrylate copolymer (b) has a (meth)acrylic acid alkyl ester as monomer unit (b3). 5) The cosmetic composition according to claim 1, further comprising at least one other component, which acts as a thickener.

6) The cosmetic composition according to claim 5, wherein the at least one component which acts as a thickener is a carbomer.

7) The cosmetic composition according to claim 1, wherein the anionic acrylate copolymer (a) is a copolymer with the INCI name Acrylates/Hydroxyesters Acrylates Copolymer, and the anionic acrylate copolymer (b) is a copolymer with the INCI name Acrylates Copolymer (and) Water.

8) The cosmetic composition according to claim 1, comprising, based on the total weight of the cosmetic composition:
a. to 1.5% by weight of the anionic acrylate copolymer (a), and
b. to 2.0% by weight of the anionic acrylate copolymer (b).

9) The cosmetic composition according to claim 1, further comprising, based on the total weight of the cosmetic composition:
a. 0.05 to 1.5% by weight of the carbomer.

10) A method for the temporary shaping of keratinic fibers, in which the cosmetic composition according to claim 1 is applied to keratinic fibers.
